(19) United States
(12) Patent Application Publication
(10) Pub. No.: US 2009/0300811 A1
(43) Pub. Date: Dec. 10, 2009

(54) PAIR OF PROTECTIVE GOGGLES

(76) Inventor: Sven-Ingvar Lundahl, Hovmantorp (SE)

Correspondence Address: DICKSTEIN SHAPIRO LLP, 1825 EYE STREET NW, Washington, DC, 20006-5403, US

(21) Appl. No.: 12/097,482

(22) PCT Filed: Dec. 12, 2006
(86) PCT No.: PCT/SE06/01418

§ 371 Date: Nov. 25, 2008

(30) Foreign Application Priority Data
Dec. 15, 2005 (SE) 0502783-4

(51) Int. Cl. A61F 9/00

(52) U.S. Cl. 2/15

(57) ABSTRACT

The present invention refers to an improvement in or relating to protective glasses (1) or a helmet having a visor, which comprises at least one supporting structure (3) having at least one translucent portion (4) in front of the eyes of an user, at least some part of the translucent portion (4) being cleanable with added liquid (5) in providing of an improved visibility through at least some part of the translucent portion (4). The added liquid (5) up to at least some extent is collectable and/or drainable into/of at least one groove (6) and/or collector (7) provided at said protective glasses (1) or said helmet having a visor.

The present invention refers to improvements in or relating to a pair of protective goggles/glasses or a helmet having a visor, which comprises a supporting structure having a translucent portion in front of the eyes of an user, at least some part of the translucent portion during wanted opportunities being cleanable with added liquid in providing of an improved visibility through the translucent portion and said added liquid is collectable or drainable into a groove or collector.

Existing structures on the market of this kind are today using different means on protective glasses or helmets having visor in order to achieve a cleaning of the translucent portion during performance of different sports such as e.g. cycling, motor cross, speedway and other sports, when dirt, clay, sand, gravel, dust and so on make the translucent portion dirty. By providing the said protective glasses with a clean washing device, which is spooling liquid on the translucent portion, the same can be cleaned. The problem is however that the liquid and dirt which is mixed together is flowing downwards over the face and clothes of the user, which moistens, cools down and make the face of the user dirty, which will be irritated. The wet and dirty clothes also are sticked to the user's body, which will be cold down. This will make the user's possibilities to perform his sport more difficult and more worse. Further, today protective glasses exist, which are completed with a protecting, translucent layer in front of the translucent portion, which mechanically by aid of rolling forwards of a foil or turning of a disc, can be fed forwards, so that a new translucent layer is achieved, when the first layer has become dirty. These structures are expensive and complicated to manufacture and to use. When the used dirty material is utilized the same must be thrown away and be replaced by a new material, which will be expensive and is time consuming. Furthermore the function is bad and often causes trouble.

The object of the present invention is to solve the problems mentioned above in that provide the translucent portion with added liquid for cleaning during wanted moments, whereby the dirty, collected liquid is collected or is drained by aid of a groove or a collector provided at the protective glasses or helmet with the visor at the same time as dirt and moisture do not irritate the user.

Thanks to the invention a well functioning device has been provided for a pair of protective glasses or a helmet with visor, which effectively make the translucent portion clean with added liquid when needed, during performance of different sports such as e.g. cycling, motor cross, speedway and other sports, when dirt, clay, sand, gravel, dust and so on bother the user, said liquid is collectable in a groove or collector and is drained, so that the user is not bothered by the collected liquid. According to a preferred embodiment example of the invention the collected liquid is fed further from said groove or collector via a drain channel to a container, which can be emptied when needed. The translucent portion in the protective glasses consists of a sheet unit such as a glass sheet or plastic sheet, which is supported by and fixed to a supporting structure, which comprises of a frame structure, such as a spectacle frame, which in turn are supported by the head of the user by aid of elastic bands or bows. The groove or collector is firmly or releaseably provided direct or indirect in connection the lower part of the translucent portion or the supporting structure. The added liquid is fed from a liquid container via an inlet into a feeding channel and is fed via said feeding channel towards the translucent portion from a number of openings in the upper part of the supporting structure towards the upper part of the translucent portion for washing and cleaning of the same. The drain channel is provided outside by aid of holders at the upper part of the protective glasses or is made as a longitudinal hollow formation within the supporting portion or the translucent portion. The groove or collector can, according to a variant of the invention, be made of the same part as the supporting structure or the translucent portion. If the translucent portion is a sheet unit the groove or collector can be created by a folding upwards of a part of said sheet unit. The feeding channel or the drain channel can in this case also be provided by bending and forming of said sheet unit. Or the bulging can be made so that it creates space for a position orientation or position fixation of said drain channel or the feeding channel in the form a separate hose or conduit. The added liquid can be fed forwards by aid of over pressure through the feeding channel, which then is ended in a number of openings such as a nozzle having a smaller diameter than the diameter of the feeding channel, so that a more effective washing is provided. The added liquid can according to the invention be pumped forwards by a hand or motor driven pump from a liquid container with the liquid, which also can be made of a resilient material, so that the liquid can be fed forwards through the feeding channel by a pressing together of said liquid container. The most important advantages of the invention are thus that a simple and cheap device for draining of collected, dirty liquid into a container has been provided, which in an easy way can be emptied when needed, so that the dirty liquid does not bother the user, which therefore such as a sport practican can perform more.

The invention is described closer below by aid of some preferred embodiment examples in view of the drawings enclosed, in which

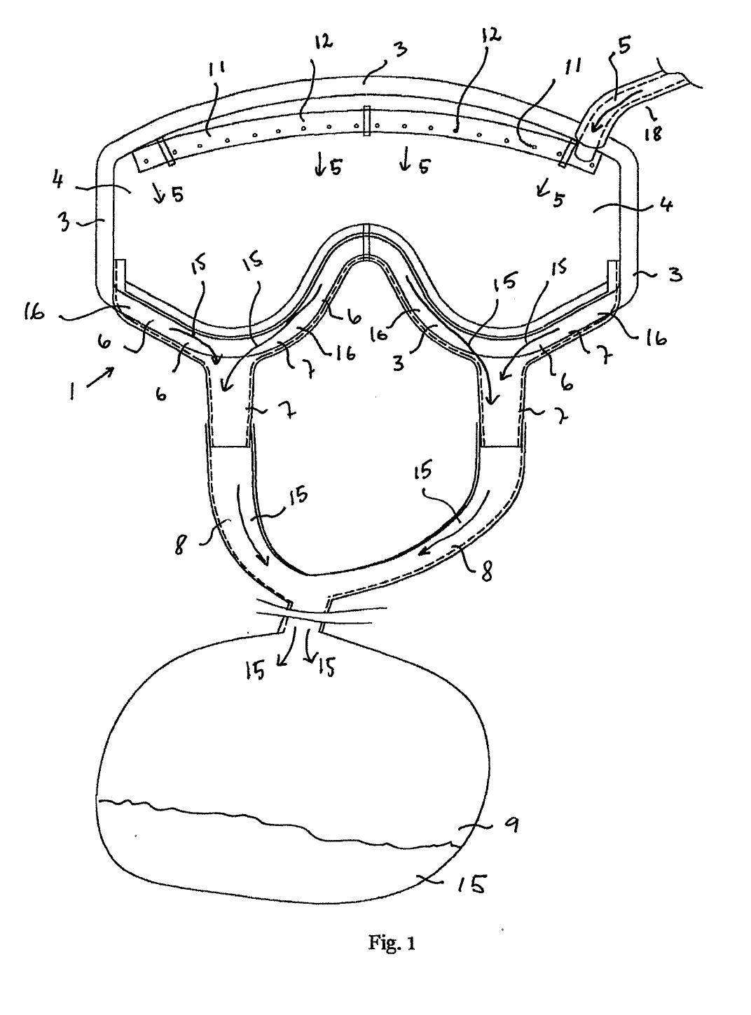

FIG. 1 shows in a front view a pair of protective glasses according to a first variant of the invention,

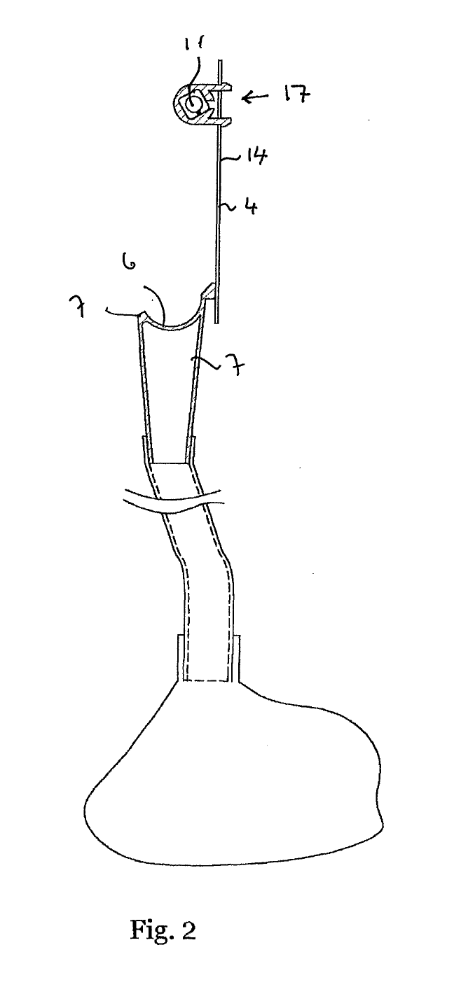

FIG. 2 shows a vertical section through a second variant of the invention,

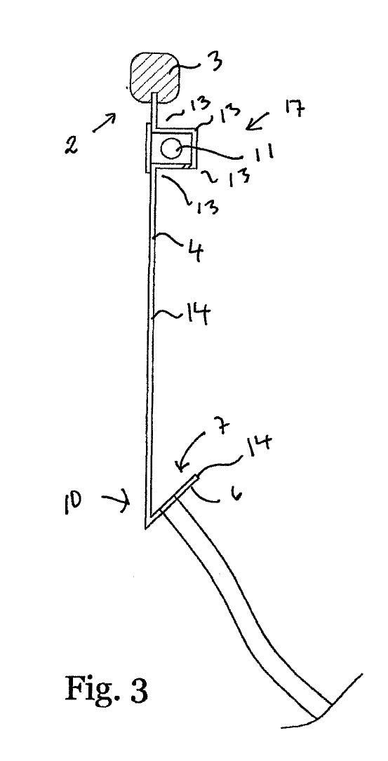

FIG. 3 shows a vertical section through a pair of protective glasses according to a third variant of the invention, and

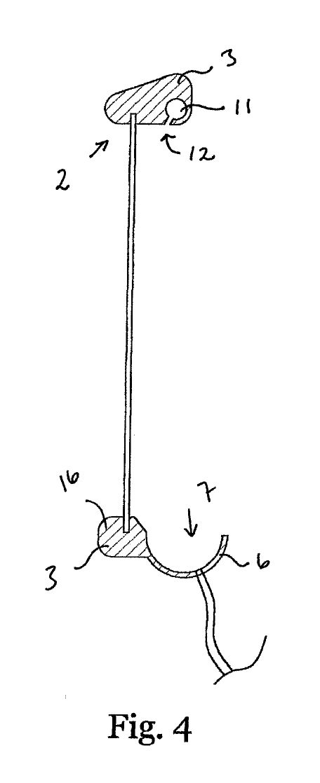

FIG. 4 shows a vertical section through a pair of protective glasses according to a fourth variant of the invention.

As can be seen from FIG. 1 and 2 here is illustrated a pair of protective glasses 1, which comprises a structure 3 for supporting of a translucent portion 4. A liquid container provides a feeding channel 11 with liquid 5 via an inlet 18. The feeding channel 11 comprises a number of openings 12, through which the fed liquid 5 is sprayed out in a direction towards the translucent portion 4 and is collected as a collected liquid 15 and is drained via grooves 6 or collectors 7, which are provided at the lower portion 16 of the supporting structure via draining channels 8 to a container 9.

As can be seen from FIG. 2, the translucent portion 4 of consists of a sheet unit 14 and the feeding channel 11 is manufactured separately and provided at the upper portion 17 of the translucent portion 4. The grooves 6 and the collectors 7 are also separately provided in said sheet unit 14.

As can be seen from FIG. 3 it is illustrated that the groove 6 or the collector 7 consists of a fold up of said sheet unit 14 at the lower portion 10 of said translucent portion 4. Furthermore a number of bulgings 13 in said sheet unit 14 are provided for a position orientation or position fixation of the feeding channel 11 at the upper portion 17 of the translucent portion 4.

As can be seen from FIG. 4 the groove 6 or the collector 7 is formed in one and the same part as the supporting structure 3. The feeding channel 11 is provided within the upper portion 2 of the supporting structure 3.

1. An improvement in or relating to protective glasses (1) or a helmet having a visor, which comprises at least one supporting structure (3) having at least one translucent portion (4) in front of the eyes of an user, at least some part of the translucent portion (4) being cleanable with added liquid (5) in providing of an improved visibility through at least some part of the translucent portion (4), characterized in that said added liquid (5) up to at least some extent is collectable and/or drainable into/of at least one groove (6) and/or collector (7) as collected liquid (15).

2. An improvement according to claim 1, characterized in that the feeding channel (11) is provided at/in an upper portion (2) located at the supporting structure (3) and/or an upper portion (17) located at the translucent portion (4)

3. An improvement according to claim 1, characterized in that the collected liquid (15) is fed forwards away from the groove (6) or collector (7) via at least one draining channel (8) to another place for drainage or collection in at least one container (9).

4. An improvement according to claim 1, characterized in that the translucent portion (4) consists of at least one sheet unit (14) such as e.g. a glass sheet or a plastic sheet provided totally or partly at and/or one upon the other.

5. An improvement according to claim 1, characterized in that the groove (6) and/or the collector (7) is fixed or loosely provided mainly directly or indirectly in connection with the lower portion (10) of the translucent portion (4) or the lower portion (16) of the supporting structure (3).

6. An improvement according to claim 1, characterized in that the added liquid (5) is fed to the translucent portion (4) through at least one feeding channel (11) provided on/at or in the supporting structure (3) and/or the translucent portion (4).

7. An improvement according to claim 1, characterized in that the groove (6) and/or the collector (7) totally or partly consists of/is formed in one and the same part as the supporting structure (3) and/or the translucent portion (4).

8. An improvement according to claim 4, characterized in that the groove (6) and/or the collector (7) totally or partly is formed by a folding up of at least one portion of the sheet unit (14).

9. An improvement according to claim 1, characterized in that the feeding channel (11) or the draining channel (8) is formed by providing of at least one bulging (13) of said sheet unit (14) or that said bulging (13) gives space for at least one position orientation and/or position fixation of said feeding channel (11) or drainage channel (8).

10. An improvement according to claim 1, characterized in that the added liquid (5) is fed via at least one inlet (18) during an over pressure in said feeding channel (11) to at least one opening (12) located in the supporting structure (3), feeding channel (11) or the translucent portion (4) in order to be sprayed out over and towards the translucent portion (4).
